(19) United States
(12) Patent Application Publication
(10) Pub. No.: US 2025/0043221 A1
(43) Pub. Date: Feb. 6, 2025

(54) Device and Method for Splitting Three-Dimensional Agglomerates

(71) Applicant: Robert Bosch GmbH, Stuttgart (DE)

(72) Inventors: Michael Stumber, Korntal-Muenchingen (DE); Carina Schuessler, Renningen (DE)

(21) Appl. No.: 18/717,791

(22) PCT Filed: Nov. 22, 2022
(86) PCT No.: PCT/EP2022/082759

§ 371 Date: Jun. 7, 2024

(30) Foreign Application Priority Data
Dec. 14, 2021 (DE) 10 2021 214 281.8

(51) Int. Cl. C12M 3/06; C12M 1/00; C12M 1/34

(52) U.S. Cl. CPC C12M 23/16; C12M 23/34; C12M 27/16; C12M 29/04; C12M 41/12

(57) ABSTRACT

Disclosed is a microfluidic device for mechanically splitting, in particular for aiding the enzymatic cleaving, of three-dimensional agglomerates into individual structures and/or agglomerate fragments, having a first fluidic connection and a second fluidic connection and at least one first microfluidic channel arranged between the first and the second fluidic connection, wherein the first microfluidic channel has at least one first retaining structure at which three-dimensional agglomerates can be mechanically split by means of friction, and wherein the at least one first retaining structure is positioned in the at least one first microfluidic channel such that unsplit three-dimensional agglomerates cannot pass any further through the microfluidic channel toward the second fluidic connection.

The present invention relates to a microfluidic device, a method for operating the same, as well as a processing unit and a cartridge comprising the microfluidic device according to the preambles of the independent claims.

### PRIOR ART

Interest in the use of three-dimensional agglomerates, e.g. organoids or spheroids for the research, diagnosis, and treatment of diseases, such as tumor diseases, has increased sharply over the past few years because such three-dimensional agglomerates may well reflect, for example, organ-specific characteristics.

Typically, these are handled manually using aids such as pipettes, reaction vessels, and laboratory equipment. In this case, microfluidics provides advantages compared to conventional laboratory tests, such as lower sample volumes and reagents required, reduced analysis times, and parallel operations.

However, given their complexity, there are numerous challenges to be overcome when implementing required process steps into a microfluidic system. One of these challenges is, for example, the cultivation and propagation of such three-dimensional cell agglomerates in a microfluidic system.

What are referred to as lab-on-a-chip systems (abbreviated as LoC systems) are microfluidic systems that accommodate functionalities of a macroscopic laboratory on a plastic substrate for an automated process. Such systems enable biochemical processes to be processed as completely as possible or to be fully automated.

Lab-on-a-chip systems typically comprise two main components. The first is a test carrier, for example in the form of a cartridge, which comprises structures and mechanisms for manipulation of a received sample, in particular passive components such as channels, reaction chambers, or upstream reagents, or also active components such as valves, pumps, or mixers.

The second main component is a control unit for controlling the microfluidic flows in the cartridge.

Wo 2016/183143 A1 describes the production of a heart cell organoid which is arranged in a microfluidic platform in order to investigate the effect of drugs on heart cells. One bioreactor of the platform has tubular stoppers on which the organoid is retained in order to hold it in a defined position.

A device for the cell culture of spheroids is known from WO 2014/179196 A1. The latter can be designed as a microfluidic device. The spheroids are cultivated in separate wells in the device. The recesses comprise retaining structures in the form of edges, which serve to stop the movement of a pipette tip in order to enable the addition or removal of a liquid medium at a defined position near the spheroid.

### DISCLOSURE OF THE INVENTION

For example, what are referred to as tumor organoids and spheroids are used to research tumor diseases. These enable a very good reproduction of various pathological tissue conditions, so they offer themselves for drug testing to evaluate the efficacy and dosage of drugs. These observations can, e.g., be used to select a personalized drug cancer therapy for the individual patient which takes individual characteristics into account and thus optimizes the efficiency of the therapy.

One way of producing tumor organoids is to remove individual cells or tissue fragments from a cancer patient's primary tumor and then cultivate them. In this case, differentiation and propagation of these cells or tissue fragments takes place, which eventually self-organize into three-dimensional structures. Therefore, the resulting tumor organoids are three-dimensional cell agglomerates that have a similar composition and architecture to that of the patient's primary tumor tissue. They have a diameter of, e.g., from 30-500 μm.

Spheroids are three-dimensional cell agglomerates that can, e.g., be generated by the aggregation and organization of several thousand cells having a diameter of 100-800 μm.

Compared to organoids, spheroids are less complex and typically comprise cells of only one type.

In order to be able to work with organoids or spheroids over a longer period of several weeks or months, established methods of 3D cell cultivation are currently used.

The main process steps are the cultivation of organoids or spheroids and their subsequent expansion. During expansion, the organoids or spheroids are enzymatically cleaved and/or mechanically split into organoid or spheroid fragments of a few tens of cells and into individual organoid cells or spheroid cells. The split organoid or spheroid fragments and cells are then reseeded. In this way, the organoids or spheroids propagate.

The term “splitting” in the context of the present invention is thus understood to mean a dissolution of connections between individual structures of a three-dimensional agglomerate, and the resulting dissociation of the three-dimensional agglomerate into agglomerate fragments and/or individual structures.

The term “three-dimensional agglomerates” in the context of the present invention is, e.g., understood to mean cell agglomerates, such as organoids or spheroids.

In a splitting process, there is, by way of example, a dissolution of connections between cells of a cell agglomerate, and the resulting dissociation of the cell agglomerate into multicellular cell agglomerate fragments or individual cells takes place.

When splitting three-dimensional agglomerates, such as organoids and spheroids, multiple steps are required to add and separate the three-dimensional agglomerates from various liquids, such as culture media, enzyme solutions or rinsing liquids such as wash buffers, with subsequent resuspension in further liquids, as well as, for example, pipetting the three-dimensional agglomerates up and down for mechanical splitting. On a laboratory scale, the vessels are changed and centrifugation steps and pipetting operations to be monitored visually are achieved. These process steps for cultivation and expansion are very labor-intensive and time-consuming and can only be performed by trained and experienced specialist personnel in appropriately equipped laboratories.

The present invention addresses a microfluidic implementation of the processes for splitting three-dimensional agglomerates, such as tumor organoids or tumor spheroids.

Provided according to the invention are a microfluidic device comprising a first fluidic connection and a second fluidic connection and at least one first microfluidic channel arranged between the first and the second fluidic connection, as well as a method for operating the same, by means of the features in the independent claims.

This is based in particular on the fact that the at least one first microfluidic channel of the microfluidic device has at least one first retaining structure on which three-dimensional agglomerates can be mechanically split by means of friction and that the at least one first retaining structure is positioned in the at least one first microfluidic channel such that it cannot be passed any further in the direction of the second fluidic connection by unsplit three-dimensional agglomerates.

The at least one first retaining structure is, e.g., designed in the form of a pillar or post.

The cross-section of the at least one first retaining structure is, for example, round or quadrangular or polygonal.

The first microfluidic channel comprises, for example, one or multiple first retaining structures. In the case of multiple first retaining structures, these are, e.g., arranged next to and/or behind one another, in particular diagonally offset one behind the other, in the at least one first microfluidic channel.

Advantageously, the microfluidic device can be used to perform the working steps for splitting three-dimensional cell agglomerates in an automated manner. As a result, much time is saved due to the elimination of manual work steps, shortened analysis times, and parallel processes. Furthermore, these work steps can therefore not only be performed by trained and experienced skilled personnel, which relieves the workload.

The automation of process steps, in turn, enables their standardization, which offers great advantages, especially for clinical and industrial applications, for example in the field of drug testing and personalized medicine.

It is further advantageous that the three-dimensional agglomerates and the agglomerate fragments, as well as the individual structures, remain viable and cultivatable.

As a result of the process steps being performed within the microfluidic device, the risk of undesirable or problematic contamination of the sample is also reduced.

It is advantageous that the at least one first retaining structure is positioned in the at least one first microfluidic channel such that it is no longer passable for three-dimensional agglomerates in the unsplit state in the direction of the second fluidic connection. In this way, it is ensured that only split agglomerate fragments and/or individual structures as well as fluids can pass through the microfluidic device further. In addition, it is advantageous that the three-dimensional agglomerates accumulate in front of the at least one retaining structure, as a result of which they are in an optimum starting position for subsequent splitting on the at least one retaining structure of the at least one microfluidic channel.

Further advantageous embodiments of the microfluidic device result from the dependent claims.

In an advantageous embodiment of the microfluidic device, the at least one first microfluidic channel splits into, in particular two, second microfluidic channels each having a smaller diameter or a smaller width and/or height than the first microfluidic channel. The fluidic resistance of the second microfluidic channels is essentially the same in order to ensure a uniform distribution of the flow and the organoid cells and/or organoid agglomerates over the second microfluidic channels.

Each second microfluidic channel comprises at least one second retaining structure. The second retaining structure is positioned in the second microfluidic channel such that it is no longer passable for agglomerate fragments which are larger than the passage between the inner wall of the second microfluidic channel and the second retaining structure and/or which are larger than the passage between two or more second retaining structures in the direction of the second fluidic connection. According to this pattern, a microfluidic channel can split into any desired number of new microfluidic channels, in particular two channels comprising a retaining structure.

The advantage thereby is that the individual method steps for splitting the three-dimensional agglomerates are performed in parallel in each of the split microfluidic channels, so that the method can be performed faster, more efficiently, and with a larger quantity of agglomerate fragments to be split. It is also advantageous that the microfluidic channels that split off have a smaller diameter than the parent channels, so that the smaller an agglomerate fragment is, the further it can pass through the microfluidic device in the direction of the second fluidic connection 1b and the more it finds optimal splitting conditions in each microfluidic channel that are adapted to its size, such as the size of the retaining structure and the dimensions of the respective microfluidic channel.

The cross section of the microfluidic channels is, e.g., rectangular or square, whereby the corners can be rounded. Alternatively, the cross section of the microfluidic channels is, e.g., round or oval.

Furthermore, in one embodiment, the first microfluidic channel can be meander-shaped.

In a further advantageous embodiment, at least one microfluidic channel narrows, in particular continuously, in the direction of the second fluidic connection. In one embodiment, for example, the first microfluidic channel, as well as all microfluidic channels that split directly or indirectly from it, thus narrow. The narrowing can also take place in stages, for example through multiple successive constrictions.

The advantage thereby is that three-dimensional agglomerates and agglomerate fragments must be split into a certain size in order to be able to pass through the channel. It is thus ensured that ultimately only agglomerate fragments of the desired size are obtained.

A further advantageous embodiment provides that the diameter, and/or the width, and/or the height of the at least one retaining structure decreases with each splitting of the microfluidic channel containing it.

The advantage thereby is that the retaining structure thus has an optimum size in relation to the size of the respective three-dimensional agglomerates and/or agglomerate fragments for the method steps for splitting these. It is thus ensured that the method is fast and efficient, and that the three-dimensional agglomerates and/or agglomerate fragments remain viable.

In one further advantageous embodiment, at least one of the microfluidic channels comprises a directional structure.

The advantage thereby is that the cell agglomerates or individual structures and/or agglomerate fragments are focused in the center of the channel and/or in the direction of the at least one retaining structure. As a result, the splitting process is accelerated and improved.

In a further advantageous embodiment, the microfluidic device comprises a heating means for controlling the temperature of at least one microfluidic channel. For the purposes of the present invention, a heating means is understood to be a means which can heat or cool the at least one microfluidic channel comprising the at least one retaining structure.

The advantage thereby is that an optimal temperature for splitting the three-dimensional agglomerates can be provided in this way.

An optimal temperature for splitting depends (among other things and for example) on the cultivation of the three-dimensional agglomerates. If organoids are cultivated

In Matrigel® (Corning), for example, it is advantageous to cool at least one microfluidic channel comprising at least one retaining structure because Matrigel liquefies at temperatures of around 4° C.

In one embodiment, in which enzymatic cleaving of the three-dimensional agglomerates is aided by mechanical splitting on the at least one retaining structure of the at least one microfluidic channel, the enzymatic cleaving can be further improved by setting an optimum effective temperature of the enzyme.

In the case of enzymatic cleaving of organoids and/or spheroids, the enzyme-containing solution comprises, e.g., the enzyme trypsin or TrypLE™ Express Enzyme (Thermofisher), whose optimal effective temperature is 370 Celsius.

In an alternative or additional embodiment, the microfluidic device comprises a means for generating and introducing ultrasound and/or a means for generating and introducing vibration into at least one microfluidic channel.

The advantage of introducing ultrasound and/or vibration is that the ultrasonic waves and/or vibrations act on the three-dimensional agglomerates in the at least one microfluidic channel to aid and improve the splitting process. Furthermore, the agglomerates being split can also be focused acoustically using ultrasound.

In a further advantageous embodiment, the microfluidic device comprises a retaining element, which is designed as a microfilter, and/or as a microsieve, and/or as a microstructured grid with pores. The pores have a diameter of 2-10 μm in particular, and preferably 3-6 μm.

The retaining element has a pore diameter smaller than the diameter of the individual structures of the three-dimensional agglomerates. The individual structures and agglomerate fragments are therefore unable to pass through the retaining element and accumulate on it.

Advantageously, fluid exchange is enabled by the retaining element, but individual structures and agglomerate fragments are retained so that they cannot unintentionally leave the microfluidic device and are therefore not lost.

Alternatively, the pores of the first retaining element have a different pore diameter adapted to the particular application.

In a further advantageous embodiment, the microfluidic device has an additional microfluidic channel which, starting from an upper side of the retaining element, opens into a third microfluidic connection.

The advantage thereby is that the individual structures and/or agglomerate fragments can thus be transported separately via the additional microfluidic channel and can be performed via the third microfluidic connection. As a result, the microfluidic device is more flexible and complex, thus enabling more flexible handling and the performance of more complex microfluidic operations.

In a further advantageous embodiment, the microfluidic device comprises at least one valve and/or at least one pump, which are in particular electrically controllable, so that the microfluidic device is electrically operable.

The advantage thereby is that at least one fluidic connection and/or at least one microfluidic channel and/or at least one reservoir for fluids can be individually closed and opened by means of one or multiple valves, so that the flow through the microfluidic device can be individually established. In this way, various ways for the feed and discharge of media, as well as the flow through the microfluidic channels, can be achieved.

In an alternative embodiment, the at least one valve is arranged outside of the microfluidic device.

In one advantageous embodiment, the feed and discharge of the fluids and/or conveyance of the fluids is performed by at least one pump. The at least one pump is, e.g., a microfluidic pump, so that the complex and complex fluidic connection of the microfluidic device to an external pump is eliminated. In this way, the fluidic feed, discharge, and conveyance processes can be easily and straightforwardly achieved, and the microfluidic device can be designed to be space-saving, portable, and mobile.

In an alternative embodiment, the at least one pump is arranged outside of the microfluidic device. The at least one pump is, e.g., a syringe pump or a peristaltic pump.

Furthermore, in one embodiment, it is advantageous for the microfluidic device to comprise at least one reservoir for a fluid. The advantage thereby is that fluids such as media, rinsing liquids, enzyme-containing solutions or staining solutions can be stored in the reservoir.

This ensures a fast and easy feed of these fluids.

The microfluidic device 10 is, e.g., accommodated on a plastic substrate or chip.

Another object of the invention is a method for mechanical splitting, in particular for aiding the enzymatic cleaving, of three-dimensional agglomerates into individual structures and agglomerate fragments by means of the microfluidic device according to the invention, said method comprising the following steps:
a) feeding a first medium comprising three-dimensional agglomerates via the first fluidic connection
b) conveying the first medium comprising three-dimensional agglomerates via the first microfluidic channel, whereby the three-dimensional agglomerates are prevented from further passage by the retaining structure.
c) pulsatile back and forth movement of the first medium comprising three-dimensional agglomerates so that the three-dimensional agglomerates are mechanically split by means of friction on the at least one retaining structure.

The phrase “back and forth movement of the first medium in a pulsatile manner” is understood to mean that the flow direction of the first medium is temporarily changed by the microfluidic device by successive intermittent forward and backward conveying of the first medium.

The advantage thereby is that the three-dimensional agglomerates come into physical contact with the at least one retaining structure of the at least one first microfluidic channel due to the pulsatile back and forth movement of the first medium and rub against it. Furthermore, the three-dimensional agglomerates come into physical contact with the inner channel walls of the at least one first microfluidic channel and rub against them. Additional friction is created between the three-dimensional agglomerates and the pulsative, back and forth movement of the first medium. This absorbs a higher velocity than the three-dimensional agglomerates, which also leads to the washing away or detachment of individual structures or agglomerate fragments on the surface of the three-dimensional agglomerates. In addition, the three-dimensional agglomerates themselves also collide, creating further friction. These frictional processes cause the connections between the individual structures, which are responsible for the cohesion of the three-dimensional agglomerate, to become increasingly loose. More and more individual structures and agglomerate fragments split off from the three-dimensional agglomerates. In this way, the three-dimensional agglomerates are mechanically dissociated. The individual structures themselves are not damaged and remain viable and cultivatable. The individual structures and agglomerate fragments can then be reseeded and cultivated and grown into three-dimensional agglomerates.

Advantageously, the microfluidic device can be used to perform the work steps for splitting three-dimensional cell agglomerates automatically. Much time is saved due to the elimination of manual work steps, shortened analysis times, and parallel processes. Furthermore, these work steps can not only be performed by trained and experienced skilled personnel, which relieves the workload.

The automation of process steps, in turn, enables their standardization, which offers great advantages, especially for clinical and industrial applications, for example in the field of drug testing and personalized medicine.

As a result of the process steps being performed within the microfluidic device, the risk of undesirable or problematic contamination of the sample is also reduced.

It is advantageous that the at least one first retaining structure is placed in the at least one first microfluidic channel such that it is not further passable towards the second fluidic port for three-dimensional agglomerates in the unsplit state. In this way, it is ensured that only split agglomerate fragments or individual structures as well as fluids can pass through the microfluidic device further. In addition, it is advantageous that the three-dimensional agglomerates accumulate in front of the at least one retaining structure, whereby they are in an optimum starting position for subsequent splitting on the at least one retaining structure of the at least one microfluidic channel.

The first medium comprising the three-dimensional agglomerates is moved back and forth through the at least one first microfluidic channel at a flow rate of, e.g., 10-80 L/s. In this case, the flow rate is, e.g., constant or varying, in particular pulsating.

Further advantageous embodiments of the method according to the invention result from the dependent claims.

In an advantageous embodiment of the method, the following step is performed after step b):
b′) feeding and conveying a first rinsing liquid via at least one first microfluidic channel for washing the three-dimensional cell agglomerates.

The first rinsing liquid removes any residues of the first medium and cleans the three-dimensional agglomerates as well as the microfluidic device.

In one embodiment, in which the mechanical splitting of the three-dimensional agglomerates is performed to aid enzymatic cleaving, a washing step by means of a first rinsing liquid is advantageous because doing so removes proteins from the first medium adhering to the three-dimensional agglomerates, which proteins otherwise inhibit, for example, the enzyme reaction.

A further advantageous embodiment provides for the following step to be performed after step b) or after step b′):
b″) supplying and conveying an enzyme-containing solution via the first microfluidic channel, and whereby in step c) the enzyme-containing solution comprising the three-dimensional agglomerates is moved back and forth in a pulsatile manner, so that the enzymatic cleaving of the three-dimensional agglomerates is mechanically aided by means of friction of these against the at least one retaining structure.

For this purpose, the enzyme-containing solution with a splitting effect is, e.g., first added to the three-dimensional agglomerates, which are then incubated with the enzyme-containing solution, e.g. for a period of ten minutes. During the incubation or subsequently, the enzyme-containing solution comprising the three-dimensional agglomerates is moved back and forth in a pulsatile manner in the at least one first microfluidic channel comprising the at least one first retaining structure in order to mechanically aid the splitting process. The enzymatic and mechanical action removes individual structures and agglomerate fragments from the three-dimensional agglomerates, so that the splitting process operation is mechanically aided, improved, and in particular accelerated.

In a further advantageous embodiment, steps b) and c), and optionally steps U) and/or b″), are also performed in microfluidic channels comprising at least one retaining structure, which separate directly or indirectly from the first microfluidic channel.

The advantage thereby is that the splitting processes are performed in parallel in each of the split-off microfluidic channels, so that the method for splitting three-dimensional agglomerates can be performed faster, more efficiently, and with a larger quantity of agglomerate fragments to be split. It is also advantageous that the splitting microfluidic channels have a smaller diameter than the parent channels so that, the smaller an agglomerate fragment is, the farther it can pass through the microfluidic device in the direction of the second fluidic connection, and the more it encounters optimal splitting conditions in each microfluidic channel that are adapted to its size, e.g. the size of the retaining structure and the dimensions of the respective microfluidic channel.

In a further advantageous embodiment, the at least one microfluidic channel comprising the at least one retaining structure is temperature-controlled. For example, a heating element is integrated into the microfluidic device for this purpose. The heating or cooling is, e.g., performed before and/or during step c) of the microfluidic method, so that the at least one microfluidic channel comprising the at least one retaining structure is at the desired temperature during the splitting process.

If enzymatic cleaving within the microfluidic device is aided by mechanical splitting on the at least one retaining structure of the at least one microfluidic channel, then the enzymatic cleaving is further improved by setting an optimum effective temperature of the enzyme.

In a further advantageous embodiment, ultrasound and/or a vibration is introduced into at least one microfluidic channel comprising the at least one retaining structure.

The advantage of introducing ultrasound and/or vibration is that these act on the three-dimensional agglomerates in the at least one microfluidic channel to aid and improve the splitting process.

In a further advantageous embodiment, the following step is performed after step c): d) conveying the split individual structures and/or agglomerate fragments via at least one microfluidic channel to a retaining element whose pores have a diameter smaller than the diameter of the split individual structures and agglomerate fragments, so that these are retained.

The individual structures and agglomerate fragments are not able to pass through the retaining element and accumulate on or in front of the retaining element. The advantage thereby is that the individual structures and agglomerate fragments are retained and cannot leave the microfluidic device unintentionally and are therefore not lost. Fluids, on the other hand, can pass through the second retaining element so that a fluid exchange can occur.

In one embodiment, it is advantageous if the split individual structures and/or agglomerate fragments are conveyed via a second rinsing liquid, which is fed to the first microfluidic channel and conveyed via this. The advantage thereby is, e.g., that the enzyme-containing solution is removed from the three-dimensional agglomerates and the splitting process is stopped in this way.

In a further advantageous embodiment, it is provided that optical and/or microscopic analysis of the split individual structures and/or agglomerate fragments can be performed on the retaining element.

The advantage thereby is that the result of the method for splitting the three-dimensional agglomerates can be analyzed directly on the retaining element. For example, the size of the agglomerate fragments can be analyzed, the amount of the individual structures can be estimated, and/or the viability of the individual structures and agglomerate fragments can be analyzed. For this purpose, the individual structures and/or agglomerate fragments are, e.g., colored on the retaining element by feeding a coloring solution. Alternatively, the three-dimensional agglomerates are colored before being fed to the microfluidic device.

Furthermore, it is advantageous in one embodiment if the following step is performed after step d):
e) returning the individual structures and/or agglomerate fragments via the at least one microfluidic channel and discharging the individual structures and/or agglomerate fragments via the first fluidic connection, which serves in this case as an outlet.

The advantage thereby is that the microfluidic device has a simple structure, and no additional components such as reservoirs or additional channels are required.

In one alternative advantageous embodiment, the following step is performed after step d):
e′) supplying a second medium via the second microfluidic connection, which serves in this case as an inlet, and guiding the second medium via a section of the first microfluidic channel to an underside of the retaining element and through this, so that the retained individual structures and/or agglomerate fragments on an upper side of the retaining element are transferred with the second medium into an additional microfluidic channel, which opens into a third fluidic connection, and discharging the individual structures and/or agglomerate fragments via the third fluidic connection, which serves in this case as an outlet.

The advantage thereby is that the individual structures and agglomerate fragments do not have to pass backwards through the at least one microfluidic channel, but are discharged via a closer, third fluidic connection. As a result, the microfluidic method for mechanical splitting of three-dimensional agglomerates is able to be repeated with new three-dimensional agglomerates, which are fed in via the first fluidic connection, whereas the individual structures and/or agglomerate fragments are discharged at regular intervals via the third fluidic connection. This prevents the retaining element from being overloaded or clogged by an excessive number of accumulating individual structures and/or agglomerate fragments.

In a particularly advantageous embodiment, the three-dimensional agglomerates are cell agglomerates, in particular organoids or spheroids, and the split individual structures are cells, in particular organoid cells or spheroid cells, and the agglomerate fragments are cell agglomerate fragments, in particular organoid fragments or spheroid fragments.

Advantageous when using cell agglomerates, e.g. organoids or spheroids, is that their viability is well maintained so that further cultivation and expansion steps are possible.

A further object of the invention is a control unit for controlling the microfluidic method according to the invention, in particular by electrical actuation of the at least one valve and/or the at least one pump.

A further object of the invention is a cartridge, in particular a microfluidic cartridge as described in, e.g., DE102016222072A1 or DE102016222075A1, comprising the microfluidic device according to the invention.

### BRIEF DESCRIPTION OF THE DRAWINGS

Embodiments of the present invention are illustrated in the drawings and further explained in the subsequent description thereof. Shown are:

FIG. 1: the schematic view of a microfluidic device according to the invention in a first embodiment with a first microfluidic channel comprising a retaining structure,

FIG. 2a: the schematic representation of a conveying of three-dimensional agglomerates in a step b) of a method according to the invention using a microfluidic device according to FIG. 1,

FIG. 2b: the schematic representation of an accumulation of organoids prior to the retaining structure in step b) of the method according to the invention using the microfluidic device according to FIG. 1,

FIG. 2c: the schematic representation of a splitting of three-dimensional agglomerates in a step c) of the method according to the invention using the microfluidic device according to FIG. 1,

FIG. 2d: the schematic representation of discharging split single structures and/or agglomerate fragments in a step e″) of the method according to the invention using a microfluidic device according to FIG. 1,

FIG. 3a: the schematic representation of a top view of a microfluidic device according to the invention in a first variant of a second embodiment comprising splitting microfluidic channels comprising retaining structures,

FIG. 3b: schematic representation of a lateral view of the microfluidic device according to the invention in a second variant of a second embodiment with splitting microfluidic channels comprising retaining structures,

FIG. 4: schematic representation of a three-dimensional technical drawing of a section of a microfluidic device according to the invention in a third embodiment comprising an additional microfluidic channel,

FIG. 5: the schematic illustration of a cartridge according to the invention comprising the microfluidic device according to FIG. 1, and

FIG. 6: schematic representation of a flow diagram of an exemplary embodiment of the method according to the invention.

### EMBODIMENTS OF THE INVENTION

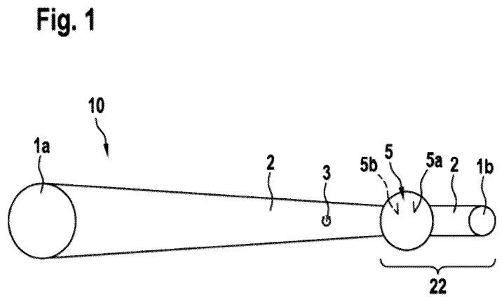

FIG. 1 shows a first embodiment of the microfluidic device 10 according to the invention for mechanically splitting three-dimensional agglomerates.

The microfluidic device 10 comprises a first fluidic connection 1a and a second fluidic connection 1b. A first microfluidic channel 2 is arranged between the first fluidic connection 1a and the second fluidic connection 1b.

The first microfluidic channel 2 comprises a first retaining structure 3, on which three-dimensional agglomerates can be mechanically split by means of friction.

The first retaining structure 3 is positioned in the first microfluidic channel 2, in particular in the center, such that the first microfluidic channel 2 is no longer passable for unsplit three-dimensional agglomerates in the direction of the second fluidic connection 1b.

The first retaining structure 3 is, e.g., designed in the shape of a pillar or post. The cross-section of the first retaining structure 3 is, e.g., round, quadrangular, or polygonal.

In one embodiment (not shown in FIG. 1), the first microfluidic channel 2 comprises, e.g., multiple first retaining structures 3. These are, e.g., arranged next to and/or behind each other in the first microfluidic channel 2, in particular diagonally offset one behind the other.

The first microfluidic channel 2 narrows continuously in the direction of the second fluidic connection 1b. In one embodiment (not shown in FIG. 1), the narrowing takes place in stages, e.g. by multiple successive narrowings.

Optionally, the microfluidic device 10 comprises a retaining element 5, which is designed as a microfilter, and/or as a microsieve, and/or as a microstructured grid with pores. The pores have a diameter of, e.g., 2-10 μm, and preferably 3-6 μm. The diameter of the pores of the retaining element 5 is thus smaller than the diameter of the agglomerate fragments and individual structures 9, so that these cannot pass through the retaining element 5 and accumulate on a top side 5a or on a bottom side 5b of the retaining element 5, (which is not visible in FIG. 1).

A section 22 of the first microfluidic channel 2 is arranged between the upper side 5a of the retaining element 5 and the second fluidic connection 1b.

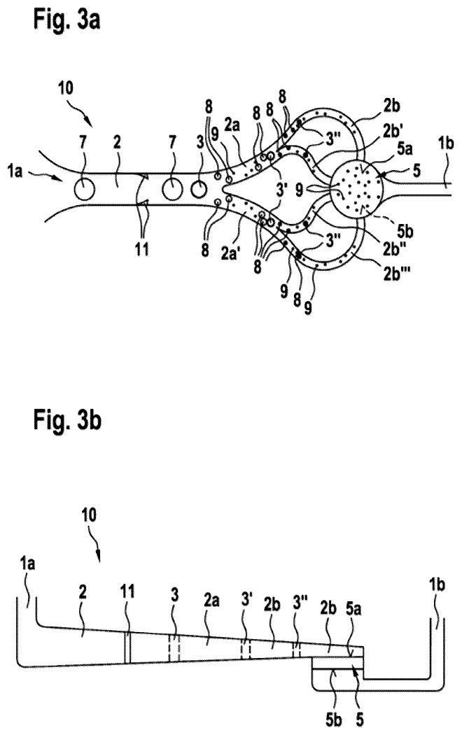

The first microfluidic channel 2 comprises, e.g., a directional structure (not shown in FIG. 1), as shown in FIG. 3a and described with respect thereto.

In a further embodiment (not shown in FIG. 1), the microfluidic device 10 comprises a heating means for controlling the temperature of the first microfluidic channel 2. Alternatively or additionally, the microfluidic device 10 comprises a means for generating and introducing ultrasound and/or a means for generating and introducing vibration into the first microfluidic channel 2.

In one embodiment, the microfluidic device 10 further comprises at least one valve (not shown in FIG. 1) and/or at least one pump (not shown in FIG. 1), which can be electrically controlled so that the microfluidic device 10 can be operated electrically. The microfluidic device 10 further comprises, e.g., at least one reservoir for a fluid (not shown in FIG. 1).

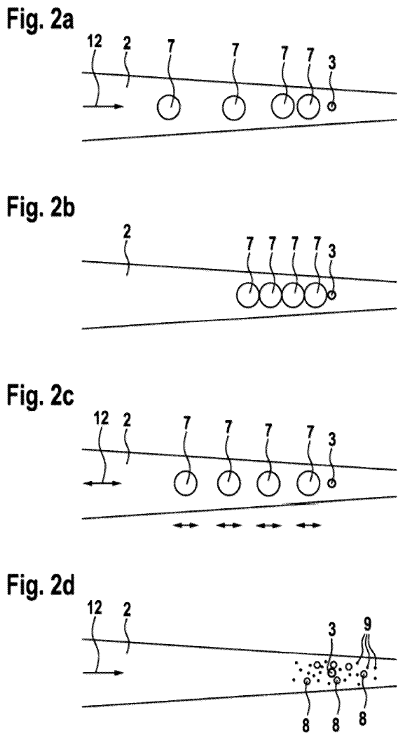

FIGS. 2a-d show a method 500 according to the invention using a microfluidic device 10 (as shown in FIG. 1), whereby FIGS. 2a-d each show a detail including the first microfluidic channel 2 and a retaining structure 3.

In the following, the mechanical splitting of three-dimensional agglomerates 7 into individual structures 9 and/or agglomerate fragments 8 is described by way of example for the mechanical splitting of organoids 7 into organoid cells 9 and/or organoid fragments 8.

In a first step a), which is not shown in FIGS. 2a-d, a first medium, e.g. a solution comprising organoids 7, is supplied to the microfluidic device 10 via the first fluidic connection 1a, which stored upstream, e.g. in a reservoir.

In a second step b), which is shown in FIG. 2a, the first medium comprising the organoids 7 is conveyed in the flow direction 12 via the first microfluidic channel 2. In this case, the organoids 7 pass through the first microfluidic channel 2 up to the first retaining structure 3, which prevents the organoids 7 from passing further in the unsplit state due to their size.

As shown in FIG. 2b, the organoids 7 accumulate behind the first retaining structure 3. The first medium, on the other hand, passes through the first retaining structure 3 and is finally discharged via the second fluidic connection 1b.

In a first embodiment of the method 500 according to the invention, the organoids 7 are split purely mechanically, without enzymatic action.

In this case, step b) is followed by step c), which is shown in FIG. 2c. The organoids 7 are mechanically split by a pulsatile back and forth movement of the first medium by means of friction on the first retaining structure 3 of the first microfluidic channel 2. The flow direction 12 is in this case changed intermittently. The friction of the organoids 7 against the first retaining structure 3 of the first microfluidic channel 2, the friction of the organoids 7 against each other against the first medium containing them, and the friction against the inner walls of the channel causes organoid cells 9 and/or organoid fragments 8 to split off from the organoids 7 until the organoids 7 are split in particular completely into organoid cells 9 and/or organoid fragments 8 consisting of a few organoid cells 9, e.g. 2-15 organoid cells 9.

There are various options for discharging the split organoid cells 9 and/or organoid fragments 8.

In a step e″), which is shown in FIG. 2d, the first medium is, e.g., supplied to the microfluidic device 10 via the first fluidic connection 1a. This medium is conveyed in the direction of flow 12 via the first microfluidic canal 2 until the second fluidic connection 1b, which is this case serves as an outlet. The organoid cells 9 and organoid fragments 8 are carried along with the first medium and discharged via the second fluidic connection 1b. In this case, a retaining element 5 can be arranged upstream of the second fluidic connection 1b, which has pores with a diameter larger than the diameter of the organoid cells 9 and smaller organoid fragments 8 of the desired size, so that these can pass through the retaining element 5.

Alternatively, the retaining element 5 can also have a pore diameter smaller than the diameter of the organoid cells 9 and organoid fragments 8, so that these cannot pass through the retaining element 5 and accumulate on its upper side 5a or lower side 5b in step d). In step e), the first medium is, e.g., supplied via the second fluidic connection 1b and conveyed through the retaining element 5 to the first fluidic connection 1a, as a result of which the organoid cells 9 and organoid fragments 8 are carried along.

In a second embodiment of the method 500 according to the invention, the mechanical splitting is performed to aid an enzymatic cleaving of the organoids 7 into organoid cells 9 and/or organoid fragments 8. The microfluidic device 10 comprises a retaining element 5 with pores smaller than the diameter of the organoid cells 9 and organoid fragments 8 so that they cannot pass through the retaining element 5.

If the organoids 7 have accumulated behind the first retaining structure 3 (as shown in FIG. 2b), then step b′) is performed after step b). In step b′), a first rinsing liquid is supplied to the microfluidic device 10, for example by switching a valve to another reservoir containing the first rinsing liquid, which is not shown in FIGS. 2a-d. The first rinsing liquid is supplied via the first fluidic connection 1a and conveyed via the first microfluidic channel 2. The first rinsing liquid washes the organoids 7, thus removing any residues from the first medium.

The first rinsing liquid is, e.g., a phosphate buffered saline (PBS). The first rinsing liquid is then discharged again via the second fluidic connection 1b.

In step b″), which is performed after step b) or after step b′), an enzyme-containing solution is supplied to the microfluidic device 10, for example by switching a valve to another reservoir containing the enzyme-containing solution, which is not shown in FIGS. 2a-d. The enzyme-containing solution is supplied via the first fluidic connection 1a and conveyed via the first microfluidic channel 2. For example, the enzyme-containing solution includes the enzyme trypsin or TrypLE™ Express Enzyme (Thermofisher), which results in enzymatic cleaving of the organoids 7.

In a next step c), the enzymatic cleaving of the organoids 7 is mechanically aided by pulsatile back and forth movement of the enzyme-containing solution comprising the organoids 7 on the first retaining structure 3 of the first microfluidic channel 2. The action of the enzyme and friction of the organoids 7 against the first retaining structure 3, friction of the organoids 7 against each other and against the enzyme-containing solution containing them and friction against the inner walls of the channel of the first microfluidic channel 2, organoid cells 9 and/or organoid fragments 8 are cleaved from the organoids 7 until the organoids 7 are completely split into organoid cells 9 and/or organoid fragments 8 from a few organoid cells 9, for example from 2-15 organoid cells 9.

Before and/or during the splitting process, the first microfluidic channel 2 comprising the first retaining structure 3 is (by way of example) heated, in particular to set an ideal temperature for the enzyme action.

Then, for example, a valve is switched to another reservoir with a second flushing liquid (not shown in the drawings).

In a next step d), the second rinsing liquid is supplied to the microfluidic device 10 via the first fluidic connection 1a and conveyed via the first microfluidic channel 2. The second rinsing liquid removes the enzyme-containing solution and thus stops the splitting process. The organoid cells 9 and organoid fragments 8 are transported via the first microfluidic channel 2 with the second rinsing liquid until they are finally retained on a lower surface 5b of the retention element 5. Only the second rinsing fluid passes through the retaining element 5 and the section 22 of the first microfluidic channel 2 and is finally discharged via the second fluidic connection 1b.

To discharge the organoid cells 9 and organoid fragments 8, a second medium is supplied to the microfluidic device 10 via the second fluidic connection 1b in step e) and conveyed via the first microfluidic channel 2 to the first fluidic connection 1a, which serves in this case as an outlet. In this case, the second medium passes through the section 22 of the first microfluidic channel 2 and the retaining element 5. The organoid cells 9 and organoid fragments 8, which have accumulated on the underside 5b of the retaining element 5, are carried along with the second medium via the first microfluidic channel 2 in step e), so that the organoid cells 9 and organoid fragments 8 are also performed via the first fluidic connection 1a.

FIG. 3a shows a top view of a microfluidic device 10 according to the invention in a first variant of the second embodiment.

In contrast to the microfluidic device 10 shown in FIG. 1, the first microfluidic channel 2 splits into two second microfluidic channels 2a, 2a′. The two second microfluidic channels 2a, 2a′ each have a smaller diameter or a smaller width and/or height than the first microfluidic channel 2. Each second microfluidic channel 2a, 2a′ comprises a second retaining structure 3′. The second retaining structures 3′ are positioned in the center of the second microfluidic channels 2a, 2a′. For agglomerate fragments 8 that are larger than the passage between the channel inner wall of the second microfluidic channel 2a, 2a′ and the second retaining structure 3′, the second microfluidic channels 2a, 2a′ cannot be passed any further in the direction of the second fluidic connection 1b.

The second microfluidic channel 2a in turn splits into two third microfluidic channels 2b, 2b′ and the second microfluidic channel 2a′ splits into two further third microfluidic channels 2b″, 2b′″.

The third microfluidic channels 2b, 2b′, 2b″, 2b′″ each have a smaller diameter or a smaller width and/or height than the second microfluidic channels 2a, 2a′.

Every third microfluidic channel 2b, 2b′, 2b″, 2b″ comprises a third retaining structure 3″. The third retaining structures 3″ are positioned centrally in the third microfluidic channels 2b, 2b′, 2b″, 2b′″ so that the third microfluidic channels 2b, 2b′, 2b″, 2b′″ for agglomerate fragments 8, which are larger than the passage between the channel inner wall of the third microfluidic channel 2b, 2b′, 2b′″, 2b′″ and the third retaining structure 3″ are no longer passable in the direction of the second fluidic connection 1b.

The third microfluidic channels 2b, 2b′, 2b″, 2b′″ each open onto an upper side 5a of the retaining element 5.

According to the pattern described, for example, a microfluidic channel 2, 2a, 2a′ splits into, in particular two, new microfluidic channels 2a, 2a′, 2b, 2b′, 2b″, 2b′″ as often as required. In FIG. 3a, the diameter or the width and/or height of the microfluidic channels 2, 2a, 2a′, 2b, 2b′, 2b″, 2b′″ are each constant. In a second variant of the second embodiment, as shown for example in FIG. 3b, the diameter and/or the width and/or the height of the microfluidic channels 2, 2a, 2a′, 2b, 2b′, 2b″, 2b′″ continuously narrows in the direction of the second fluidic connection 1b.

The diameter of the retaining structures 3, 3′, 3″ decreases with each splitting of the microfluidic channel 2, 2a, 2a′, 2b, 2b′, 2b″, 2b′″. In an alternative embodiment (not shown in the drawings), the diameter or width and/or height of the retaining structures 3, 3′, 3″ is the same in all microfluidic channels 2, 2a, 2a′, 2b, 2b′, 2b″, 2b′″. In a further alternative embodiment (not shown in the drawings), the diameter and/or the width and/or the height of the microfluidic channels 2, 2a, 2a′, 2b, 2b′, 2b″, 2b′″ gradually narrows in the direction of the second fluidic connection 1b, e.g. by multiple successive constrictions.

The first microfluidic channel 2 comprises a directional structure 11 for focusing the three-dimensional agglomerates 7 or individual structures 9 and/or agglomerate fragments 8 into the center of the channel in the direction of the at least one retaining structure (3, 3′, 3″). Alternatively, for example, each microfluidic channel 2, 2a, 2a′, 2b, 2b′, 2b″, 2b′″ has a directional structure 11.

FIG. 3b shows a lateral view of the microfluidic device 10 according to the invention in a second variant of the second embodiment. This corresponds to the first variant of the second embodiment shown in FIG. 3a, with the difference that the microfluidic channels 2, 2a, 2a′, 2b, 2b″, 2b′″, 2b′″ continuously taper in the direction of the second fluidic connection 1b.

In the side view, the first microfluidic channel 2, the second microfluidic channel 2a and the third microfluidic channel 2b are visible, each of which splits off from the other, as shown in FIG. 3A.

The microfluidic device 10 in the second embodiment is designed to perform a method 500 for mechanically splitting three-dimensional agglomerates 7 into individual structures 9 and agglomerate fragments 8 and is described below by way of example for a mechanical splitting of organoids 7 into organoid cells 9 and organoid fragments 8.

The method 500 is performed in a manner similar to the method 500 comprising the retaining element 5 described regarding FIGS. 2a-d, the pores of which element have a smaller diameter than the diameter of the organoid cells 9 and organoid fragments 8. In contrast to the latter, the microfluidic channels split. The organoid cells 9 and organoid fragments 8 which were split at the first retaining structure 3 of the first microfluidic channel 2 in step c) pass through the first microfluidic channel 2 and are then divided into the two second microfluidic channels 2a, 2a′. In the second microfluidic channels 2a, 2a′, the organoid fragments 8, which are larger than a passage between the channel inner walls and the second retaining structure 3′, are retained by the second retaining structure 3′ and accumulate in front of it. In a further step c), the retained organoid fragments 8 are split into organoid cells 9 and small organoid fragments 8. The individual structures 9 and small organoid fragments 8 then fit through the passage between the inner channel walls of the second microfluidic channels 2a, 2a′ and the second retaining structure 3′ and continue to pass through the second microfluidic channels 2a, 2a′ in the direction of the second fluidic connection 1b. The second microfluidic channels 2a, 2a′ branch off further into third microfluidic channels 2b, 2b′, 2b″, 2b′″ comprising third retaining structures 3″. The organoid cells 9 and small agglomerate fragments 8 are divided between the four third microfluidic channels 2b, 2b′, 2b″, 2b′″. In the third microfluidic channels 2b, 2b′, 2b″, 2b′″ the small agglomerate fragments 8, which are larger than a passage between the channel inner walls of the third microfluidic channels 2b, 2b′, 2b″, 2b′″ and the third retaining structure 3″ are retained by the third retaining structure 3″ and accumulate in front of it. In a further step c), the retained small organoid fragments 8 are split into organoid cells 9 and very small organoid fragments 8. The organoid cells 9 and very small organoid fragments 8 then fit through the passage between the inner channel walls of the third microfluidic channels 2b, 2b′, 2b″, 2b′″ and the third retaining structure 3′″ and can continue to pass through the third microfluidic channels 2b, 2b′, 2b″, 2b′″ towards the second fluidic port 1b. The third microfluidic channels 2b, 2b′, 2b″, 2b′″ open on the upper side 5a of the retaining element 5, on which the organoid cells 9 and very small organoid fragments 8 accumulate. Here, the organoid cells 9 and very small organoid fragments 8 are subjected to optical analysis, for example.

The organoid cells 9 and very small organoid fragments 8 are, e.g., discharged as described in the second embodiment of the method according to the invention 500 in FIG. 2.

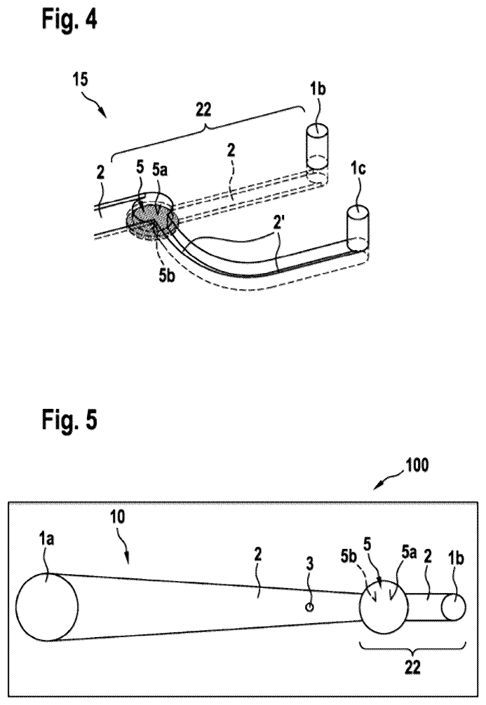

FIG. 4 shows a partial area 15 of a microfluidic device 10 according to the invention in a third embodiment. This partial area 15 is, e.g., integrated into the microfluidic device 10 according to the invention in the first embodiment (which is shown in FIG. 1), or in the microfluidic device 10 according to the invention in the second embodiment (shown in FIGS. 3a and 3b).

By means of a device 10 with an integrated section 15, it is possible to alternatively discharge the individual structures 9 and agglomerate fragments 8 retained on the upper side 5a of the retaining element 5 via an additional microfluidic channel 2′, which, starting from an upper side 5a of the retaining element 5, opens into a third fluidic connection 1c.

To discharge the organoid cells 9 and organoid fragments 8, the first microfluidic channel 2 adjacent to the retaining element 5 in the direction of the first fluidic connection 1a is, e.g., closed by a valve (not shown), and the additional microfluidic channel 2′ is, e.g., opened by opening a valve (not shown).

In step e′), a second medium is supplied via the second fluidic connection 1b, which serves in this case as an inlet. The second medium is conveyed via the region 22 of the first microfluidic channel 2, passes the retaining element 5, flowing from a lower side 5b of the retaining element 5 through the latter to an upper side 5a. The organoid cells 9 and organoid fragments 8, which have accumulated on the upper side 5a of the retaining element 5, are carried along with the second medium, fed into the additional microfluidic channel 2′ and discharged via the third fluidic connection 1c, which serves in this case as an outlet.

In all of the embodiments of the method 500 according to the invention described, the discharged organoid cells 9 and agglomerate fragments 8 can be analyzed, forwarded, divided, and/or reseeded for a repeated cultivation of organoids 7.

For example, similar to the embodiments of the method 500 for splitting organoids 7 according to the invention described, a method 500 for splitting spheroids into spheroid cells and/or spheroid fragments, in particular for aiding enzymatic cleaving, is performed.

FIG. 5 shows a cartridge 100 according to the invention, which, by way of example, comprises all embodiments of the microfluidic device 10 according to the invention, a microfluidic device 10 in the first embodiment according to FIG. 1.

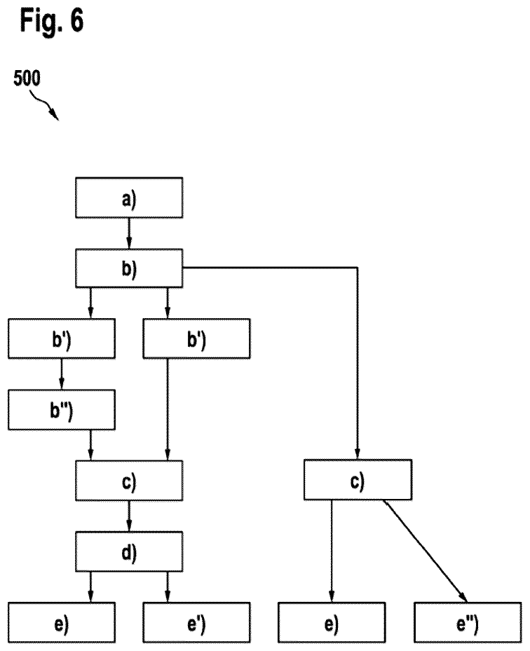

FIG. 6 shows a flow diagram with exemplary embodiments of the method 500 according to the invention for mechanical splitting, in particular for aiding the enzymatic cleaving, of three-dimensional agglomerates 7 into individual structures 9 and/or agglomerate fragments 8, for example in conjunction with the exemplary embodiments and method steps described with respect to FIGS. 1-4.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. A microfluidic device for mechanical splitting for aiding the enzymatic cleaving of three-dimensional agglomerates into individual structures and/or agglomerate fragments, comprising:
a first fluidic connection;
a second fluidic connection; and
at least one first microfluidic channel arranged between the first fluidic connection and the second fluidic connection,
wherein
the first microfluidic channel has at least one first retaining structure at which three-dimensional agglomerates are mechanically split by friction, and
the at least one first retaining structure is positioned in the at least one first microfluidic channel such that unsplit three-dimensional agglomerates cannot pass any further through the microfluidic channel toward the second fluidic connection.

23. The microfluidic device according to claim 22, wherein:
the at least one first microfluidic channel splits into two second microfluidic channels each having a smaller diameter, or a smaller width, and/or a smaller height than the first microfluidic channel, and
each second microfluidic channel comprises at least one second retaining structure which is positioned in the second microfluidic channel such that agglomerate fragments which are larger than a passage between an inner wall of the second microfluidic channel and the second retaining structure and/or which are larger than a passage between second retaining structures, cannot pass in the direction of the second fluidic connection, and
a microfluidic channel splits into two new microfluidic channels comprising a retaining structure as often as required according to this pattern.

24. The microfluidic device according to claim 22, wherein:
at least one microfluidic channel narrows continuously in the direction of the second fluidic connection.

25. The microfluidic device according to claim 22, wherein:
the microfluidic device comprises a heating device configured to control the temperature of at least one microfluidic channel, and/or a means for generating and introducing ultrasound, and/or a means for generating and introducing vibration into at least one microfluidic channel.

26. The microfluidic device according to claim 22, wherein:
the microfluidic device comprises a retaining element which is designed as a microfilter, and/or as a microsieve, and/or as a microstructured grid with pores; and
the pores have a diameter of 2-10 μm.

27. The microfluidic device according to claim 26, wherein:
the microfluidic device comprises an additional microfluidic channel which, starting from an upper side of the retaining element, opens into a third microfluidic connection.

28. A microfluidic method for mechanical splitting for aiding enzymatic cleaving of three-dimensional agglomerates into individual structures and/or agglomerate fragments using the microfluidic device according to claim 22, comprising:
a) feeding a first medium comprising three-dimensional agglomerates via the first fluidic connection;
b) conveying the first medium comprising three-dimensional agglomerates via the first microfluidic channel, wherein the three-dimensional agglomerates are prevented from further passage by the at least one first retaining structure; and
c) moving the first medium comprising three-dimensional agglomerates in a pulsatile back and forth movement so that the three-dimensional agglomerates are mechanically split by friction on the at least one first retaining structure.

29. The microfluidic method according to claim 28, wherein:
b′) feeding and conveying an enzyme-containing solution via the first microfluidic channel is performed after step b); and
in step c) the enzyme-containing solution comprising the three-dimensional agglomerates is moved back and forth in a pulsatile manner such that the enzymatic cleaving of the three-dimensional agglomerates is mechanically aided by friction on the at least one first retaining structure.

30. The microfluidic method according to claim 28, wherein steps b) and c), are also performed in microfluidic channels, comprising at least one retaining structure, which separate directly or indirectly from the first microfluidic channel.

31. The microfluidic method according to claim 28, wherein:
at least one microfluidic channel comprising the at least one retaining structure is temperature-controlled by heating or cooling, and/or
ultrasound and/or a vibration is introduced into at least one microfluidic channel comprising the at least one retaining structure.

32. The microfluidic method according to claim 28, wherein:
d) conveying the split individual structures and/or agglomerate fragments via at least one microfluidic channel to a retaining element, the pores of which have a diameter smaller than the diameter of the split individual structures and/or agglomerate fragments so that these are retained is performed after step c); and
the conveyance of the split individual structures and/or agglomerate fragments is performed via a second rinsing liquid.

33. The microfluidic method according to claim 28, wherein:
e) returning the individual structures and/or agglomerate fragments via at least one microfluidic channel and discharging the individual structures and/or agglomerate fragments via the first fluidic connection which serves as an outlet, is performed after step d).

34. The microfluidic method according to claim 32, wherein:
e′) supplying a second medium via the second microfluidic connection which serves as an inlet, and guiding the second medium via a section of the first microfluidic channel to an underside of the retaining element and through the latter so that the retained individual structures and/or agglomerate fragments on an upper side of the retaining element are transferred with the second medium into an additional microfluidic channel which opens into a third fluidic connection, and discharging the individual structures and/or agglomerate fragments via the third fluidic connection which serves as an outlet, is performed after step d).

35. The microfluidic method according to claim 28, wherein:
the three-dimensional agglomerates are cell agglomerates in the form of organoids or spheroids;
the split individual structures are cells in the form of organoid cells or spheroid cells; and
the agglomerate fragments are cell agglomerate fragments in the form of organoid fragments or spheroid fragments.

36. A cartridge in the form of a microfluidic cartridge, comprising a microfluidic device according to claim 22.
